(19) United States
(12) Patent Application Publication
(10) Pub. No.: US 2009/0012127 A1
(43) Pub. Date: Jan. 8, 2009

(54) Thiophene-2-Carboxamide Derivatives as Alpha 7 Nicotinic Receptor Modulators

(75) Inventors: Thomas R. Simpson, Wilmington, DE (US); Michael Balestra, Wilmington, DE (US); Dean G. Brown, Wilmington, DE (US); Cathy L. Dantzman, Wilmington, DE (US); Glen E. Ernst, Wilmington, DE (US); William Frietze, Wilmington, DE (US); Christopher R. Holmquist, Wilmington, DE (US); James Kang, Wilmington, DE (US); Frances M. McLaren, Wilmington, DE (US); Reed W. Smith, JR., Wilmington, DE (US); James M. Woods, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Södertälje (SE)

(21) Appl. No.: 11/917,818

(22) PCT Filed: Jun. 26, 2006
(86) PCT No.: PCT/SE2006/000776

§ 371 Date: Apr. 8, 2008

Related U.S. Application Data
(60) Provisional application No. 60/694,842, filed on Jun. 29, 2005.

(51) Int. Cl. A61K 31/4436; A61K 31/4245; A61K 31/4155; A61K 31/381; C07D 409/02; A61P 25/18; A61P 25/16; A61P 25/00; A61P 25/34; A61P 25/28; C07D 333/32; A61K 31/4025; A61K 31/422

(52) U.S. Cl. 514/336; 549/64; 514/445; 546/280.4; 548/247; 514/378; 514/364; 548/126; 548/365.7; 514/406; 514/414; 548/527; 548/255

(57) ABSTRACT

Compounds of Formula (I) wherein E, A and R1 are as described in the specification, pharmaceutically-acceptable salts, methods of making, pharmaceutical compositions containing and methods for using the same.

### FIELD OF THE INVENTION

The present invention relates to positive modulators of nicotinic acetylcholine receptors, such positive modulator having the capability to increase the efficacy of nicotinic receptor agonists. The invention particularly relates to compounds or pharmaceutically-acceptable salts thereof, processes for preparing them, pharmaceutical compositions containing them and their use in therapy.

### BACKGROUND OF THE INVENTION

Cholinergic receptors normally bind the endogenous neurotransmitter acetylcholine (ACh), thereby triggering the opening of ion channels. ACh receptors in the mammalian central nervous system can be divided into muscarinic (mAChR) and nicotinic (nAChR) subtypes based on the agonist activities of muscarine and nicotine, respectively. The nicotinic acetylcholine receptors are ligand-gated ion-channels containing five subunits. Members of the nAChR subunit gene family have been divided into two groups based on their amino acid sequences; one group containing so-called P subunits, and a second group containing ox subunits. Three kinds of β subunits, α7, α8 and α9, have been shown to form functional receptors when expressed alone and thus are presumed to form homooligomeric pentameric receptors.

An allosteric transition state model of the nAChR has been developed that involves at least a resting state, an activated state and a “desensitized” closed channel state, a process by which receptors become insensitive to the agonist. Different nAChR ligands can stabilize the conformational state of a receptor to which they preferentially bind. For example, the agonists ACh and (−)-nicotine respectively stabilize the active and desensitized states.

Changes of the activity of nicotinic receptors has been implicated in a number of diseases. Some of these, for example myasthenia gravis and ADNFLE (autosomal dominant nocturnal front lobe epilepsy) are associated with reductions in the activity of nicotinic transmission either because of a decrease in receptor number or increased desensitization. Reductions in nicotinic receptors have also been hypothesized to mediate cognitive deficits seen in diseases such as Alzheimer's disease and schizophrenia.

The effects of nicotine from tobacco are also mediated by nicotinic receptors. and since the effect of nicotine is to stabilize receptors in a desensitized state, an increased activity of nicotinic receptors may reduce the desire to smoke.

Compounds which bind nACHrs have been suggested for the treatment of a range of disorders involving reduced cholinergic function such as Alzheimer's disease, cognitive or attention disorders, attention deficit hyperactivity disorders, anxiety, depression, smoking cessation, neuroprotection, schizophrenia, analgesia, Tourette's syndrome, and Parkinson's disease.

However, treatment with nicotinic receptor agonists which act at the same site as ACh is problematic because ACh not only activates, but also blocks receptor activity through processes which include desensitization and uncompetitive blockade. Furthermore, prolonged activation appears to induce a long-lasting inactivation. Therefore, agonists of ACh can be expected to reduce activity as well as enhance it.

At nicotinic receptors in general, and of particular note at the α7-nicotinic receptor, desensitization limits the duration of action of an applied agonist.

### SUMMARY OF THE INVENTION

We have found that certain thiophene amide compounds can positively modulate the action of agonists at nicotinic acetylcholine receptors (nAChR). Such modulators increase the efficacy of agonists. Compounds having this type of action are those of Formula I:

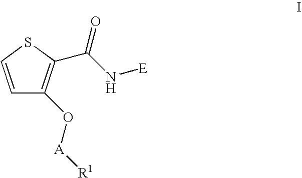

wherein:

E is selected from a moiety of formula II or III,

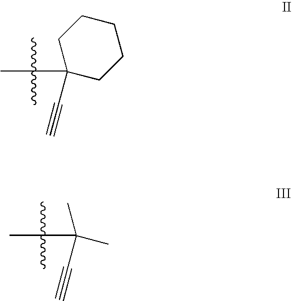

A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and

R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:

R¹ may be unsubstituted or be substituted with 1 or 2 moieties selected from CN, phenyl, halogen, —C₁₋₄alkyl and —C₁₋₄alkoxy.

Also described are stereoisomers, enantiomers, in vivo-hydrolysable precursors and pharmaceutically-acceptable salts of compounds of formula I, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

Compounds described herein are positive modulators likely to be particularly useful for treatment of conditions associated with reductions in nicotinic transmission. In a therapeutic setting such compounds could restore normal interneuronal communication without affecting the temporal profile of activation. In addition, positive modulators are not expected to produce long-term inactivation of receptors as may the prolonged application of agonists.

### DETAILED DESCRIPTION OF THE INVENTION

In one aspect the invention encompasses compounds of Formula I:

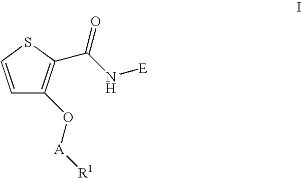

wherein:

E is selected from a moiety of formula II or III,

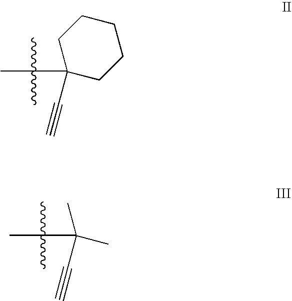

A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and

R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:

R¹ may be unsubstituted or be substituted with 1 or 2 moieties selected from CN, phenyl, halogen, —C₁₋₄alkyl and —C₁₋₄alkoxy,

stereoisomers, enantiomers, in vivo-hydrolysable precursors and pharmaceutically-acceptable salts thereof.

A particular aspect the invention encompasses compounds of Formula IV:

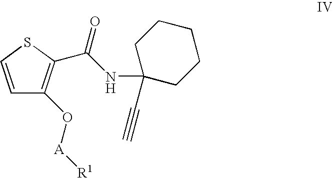

wherein:

A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and

R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:

R¹ may be unsubstituted or be substituted with 1 or 2 moieties selected from CN, phenyl, halogen, —C₁₋₄alkyl and —C₁₋₄alkoxy,

stereoisomers, enantiomers, in vivo-hydrolysable precursors and pharmaceutically-acceptable salts thereof.

Yet another particular aspect described herein encompasses compounds of Formula V:

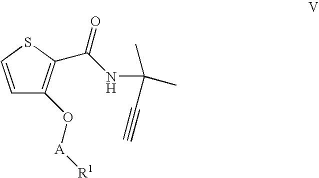

wherein:

A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and

R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:

R¹ may be unsubstituted or be substituted with 1 or 2 moieties selected from CN, phenyl, halogen, —C₁₋₄alkyl and —C₁₋₄alkoxy,

stereoisomers, enantiomers, in vivo-hydrolysable precursors and pharmaceutically-acceptable salts thereof.

A most particular aspect described herein are those compounds described herein.

In another aspect the invention is a method of treatment or prophylaxis of psychotic disorders, intellectual impairment disorders or diseases or conditions in which modulation of the α7 nicotinic receptor is beneficial, which method comprises administering a therapeutically-effective amount of a positive modulator of Formula I, IV or V as described above or a diastereoisomer, enantiomer or pharmaceutically-acceptable salt thereof.

A particular aspect of the method described herein is a method of treatment for Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, inflammatory bowel disease, ulcerative colitis or irritable bowel syndrome.

Methods of treatment of this invention include administering either a positive modulator as the only active substance, thus modulating the activity of endogenous nicotinic receptor agonists such as acetylcholine or choline, or administering a positive modulator together with a nicotinic receptor agonist.

In a particular form of this aspect, the method of treatment comprises treatment with an α7-nicotinic receptor modulator as described herein and an α7-nicotinic receptor agonist. An example of a suitable α7-nicotinic receptor agonist is (−)-spiro[1-azabicyclo[2.2.2.]octane-3,5′-oxazolidine]-2′-one. Other α7-nicotinic receptor agonists useful for treatment in conjunction with positive modulators of the present invention are described in international publications WO 96/06098, WO 97/30998 and WO 99/03859.

Another aspect described herein comprises methods of preparing compounds according to Formula I, IV or V.

Positive modulators described herein have the advantage that they are less toxic, more efficacious, longer acting, have a broader range of activity, be more potent, produce fewer side effects, are more easily absorbed or have other useful pharmacological properties.

Acid addition salts are also within the scope described herein. Such salts include salts of mineral acids, for example the hydrochloride and hydrobromide salts; and salts formed with organic acids such as formate, acetate, maleate, benzoate, tartrate, and fumarate salts. Acid addition salts of compounds of Formula I, IV or V may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

The compounds of Formula I, IV or V may exist in tautomeric or enantiomeric forms, all of which are included within the scope described herein. The various optical isomers may be isolated by separation of a racemic mixture of the compounds using conventional techniques, for example by fractional crystallization, or chiral HPLC. Alternatively the individual enantiomers may be made by reaction of the appropriate optically active starting materials under reaction conditions which will not cause racemization.

A further aspect described herein comprises a pharmaceutical composition for treating or preventing a condition or disorder as described herein arising from dysfunction of nicotinic acetylcholine receptor neurotransmission in a mammal, preferably a human. Such a pharmaceutical composition comprises a therapeutically-effective amount of a compound of Formula I, IV or V, an enantiomer thereof or a pharmaceutically-acceptable salt thereof, effective in treating or preventing such disorder or condition and a pharmaceutically-acceptable carrier.

Another aspect described herein is a pharmaceutical composition comprising a compound according to Formula I, IV or V as described herein or a diastereoisomer, enantiomer or pharmaceutically-acceptable salt thereof, together with at least one pharmaceutically-acceptable diluent or carrier.

In particular, this aspect provides a pharmaceutical composition including preferably less than 80% and more preferably less than 50% by weight of a compound described herein in admixture with a pharmaceutically-acceptable diluent or carrier.

Examples of diluents and carriers are:
for tablets and dragees: lactose, starch, talc, stearic acid;
for capsules: tartaric acid or lactose;
for injectable solutions: water, alcohols, glycerin, vegetable oils;
for suppositories: natural or hardened oils or waxes.

Yet another pharmaceutical composition described herein comprises in addition a nicotinic receptor agonist.

Another aspect described herein provides a process for the preparation of a pharmaceutical composition, which comprises incorporating the ingredients in a composition by conventional processes.

Yet a further aspect described herein is the use of a compound according to Formula I, IV or V, an enantiomer thereof or a pharmaceutically-acceptable salt thereof, for the preparation of a medicament.

A particular aspect described herein is the use of a compound according to Formula I, IV or V as described herein or a diastereoisomer, enantiomer or pharmaceutically-acceptable salt thereof, in the manufacture of a medicament for the treatment or prophylaxis of psychotic disorders, intellectual impairment disorders, human diseases or conditions in which modulation of the α7 nicotinic receptor is beneficial including Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, ulcerative colitis or irritable bowel syndrome.

In a particular form, this aspect is the use of compound according to the invention in the manufacture of a medicament for the treatment or prophylaxis of a condition associated with reduced nicotinic receptor transmission or a condition associated with reduced nicotinic receptor density which could be one of the diseases or conditions mentioned herein, which treatment comprises administering said medicament comprising a therapeutically effective amount of a compound according to the invention to a patient.

It will be understood that this use includes the manufacture of medicaments comprising either a positive modulator as the only active substance providing modulation of the activity of endogenous nicotinic receptor agonists, or the manufacture of medicaments comprising a positive modulator in combination with a nicotinic receptor agonist. Thus, this use provides for the manufacture of medicaments containing a positive modulator and medicaments containing in addition a nicotinic receptor agonist.

In a particular form of this aspect, the medicament or pharmaceutical composition comprises an α7-nicotinic receptor modulator as described herein and an α7-nicotinic receptor agonist. An example of a suitable α7-nicotinic receptor agonist is (−)-spiro[1-azabicyclo[2.2.2.]octane-3,5′-oxazolidine]-2′-one. Other α7-nicotinic receptor agonists useful in medicaments in conjunction with positive modulators of the present invention are described in international publications WO 96/06098, WO 97/30998 and WO 99/03859.

Still a further aspect described herein is a method of treating or preventing a condition or disorder in mammals and particularly humans as mentioned herein arising from dysfunction of nicotinic acetylcholine receptor neurotransmission.

A particular form of this aspect provides a method for the treatment of a condition associated with reduced nicotine transmission, by administering to a patient in need of such treatment, a medically effective amount of a positive modulator of a nicotinic receptor agonist, said positive modulator having the capability to increase the efficacy of the said nicotinic receptor agonist.

In the above-mentioned compositions, uses and methods, the amount of a compound according to Formula I, IV or V employed will, of course, vary with the compound employed, the mode of administration and the treatment desired. However, in general, satisfactory results will be obtained when a compound described herein is administered to provide a daily dosage of from about 0.1 mg to about 20 mg per kg of animal body weight, which may be given as divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carrier or diluent.

In compositions, uses and methods described herein, a compound of Formula I, IV or V, an enantiomer thereof, or a pharmaceutically-acceptable salt thereof, may be used on its own in the form of appropriate medicinal preparations for enteral or parenteral administration or may be used in a composition containing other pharmacologically-active agents. For example, a composition containing other pharmacologically-active agents may contain a positive modulator compound according to Formula I, IV or V together with a nicotinic receptor agonist.

It will be understood that the a positive modulator described herein can be administered either with the purpose of modulating the action of endogenous nicotine receptor agonists such as acetylcholine or choline, or to modulate the action of an exogenous nicotinic receptor agonist.

Accordingly, the invention includes compositions comprising a positive modulator as the only active substance, thus modulating the activity of endogenous nicotinic receptor agonists such as acetylcholine or choline; and compositions comprising a positive modulator in combination with a nicotinic receptor agonist. Thus, the said pharmaceutical compositions containing a positive modulator of a nicotinic receptor agonist may in addition comprise a nicotinic receptor agonist.

Examples of diseases or conditions for which aspects of the present invention are contemplated to be useful include schizophrenia, mania and manic depression, anxiety, Alzheimer's disease, learning deficit, cognition deficit, attention deficit, memory loss, Lewy Body Dementia, Attention Deficit Hyperactivity Disorder, Parkinson's disease, Huntington's disease, Tourette's syndrome, jetlag, and nicotine addiction (including that resulting from exposure to products containing nicotine).

### Biological Methods

The activity of the compounds described herein may be measured in the tests set out below:

### (a) Xenopus Oocyte Current Recording

Xenopus oocytes provide a powerful means of assessing the function of proteins thought to be subunits of ligand-gated ion-channels. Injection of RNA transcribed from cDNA clones encoding the appropriate receptor subunits, or injection of cDNA in which the coding sequence is placed downstream of a promoter, results in the appearance of functional ligand-gated ion-channels on the surface of an oocyte (see e.g. Boulter et al. (1987) Proc. Natl. Acad. Sci. U.S.A. 84, 7763-7767).

Consequently, one convenient technique to assess the enhancement of nicotinic efficacy is two-electrode voltage-clamp recording from Xenopus oocytes that express α7-nicotinic receptors from cRNA.

Xenopus laevis frogs (Xenopus I, Kalamazoo, Mich.) may be anesthetized using 0.15% tricaine. Oocytes are removed to OR2 solution (82 mM NaCl, 2.5 mM KCl, 5 mM HEPES, 1.5 mM NaH₂PO₄, 1 mM MgCl₂, 0.1 mM EDTA; pH 7.4). The oocytes are defolliculated by incubation in 25 mL OR2 containing 0.2% collagenase 1A (Sigma) two times for 60 min on a platform vibrating at 1 Hz and may be stored in Leibovitz's L-15 medium (50 μg/ml gentomycin, 10 Units/ml penicillin, and 10 μg/ml streptomycin). Approximately 50 ng of cRNA is injected into each oocyte on the following day.

Oocytes are placed in an external recording solution consisting of 90 mM NaCl, 1 mM KCl, 1 mM MgCl₂, 1 mM BaCl₂, 5 mM HEPES at pH 7.4. Two-electrode voltage-clamp recording may be carried out using an Oocyte Clamp amplifier (for example an OC 725C; Warner Instrument, Hamden, Conn.). Oocytes are impaled with two electrodes of 1-2 MΩ tip resistance filled with 3M KCl. Recordings are begun when membrane potential becomes stable at potentials negative to −20 mV (resting membrane potentials are less negative when Ba⁺⁺ replaces Ca⁺⁺ in bathing solutions). Membrane potential is clamped at −80 mV. Oocytes are continuously perfused at 5 mL/min with a recording solution with or without acetylcholine.

Current amplitude is measured from baseline to peak. EC₅₀ values, maximal effect, and Hill slopes may be estimated by fitting the data to the logistic equation using, for example, GraphPad Prism (GraphPad Software, Inc., San Diego, Calif.).

Increases in agonist efficacy elicited by a positive modulator can be calculated in two ways:

(1) As a percent potentiation of current amplitude which is defined as 100(Im−Ic)/Ic where Im is current amplitude in the presence of modulator and Ic is current in the absence of modulator.

(2) As a percent potentiation of “area under curve” of an agonist trace, which is the integration of net current over time. Area under the curve is used to represent of the total ion flux through the channel.

### (b) Ca⁺⁺ Flux Imaging

Imaging of Ca⁺⁺ flux through nAChR α7 receptors transiently expressed in a cell line is another means of assaying modulator activity.

Cells expressing α7 receptors (for example HEK-293 cells or cell-cultured neurons) are grown to confluence in 96 well plates and loaded with fluo-3, a fluorescent calcium indicator. To screen for α7 modulatory activity, a 96 well plate is placed in a fluorescence imaging plate reader (FLIPR) and test compounds along with an α7 agonist are applied simultaneously to all wells. Receptor activation is measured by calcium influx into cells which is quantified by the increase in fluorescence intensity of each well, as recorded simultaneously by the FLIPR. A modulatory effect is shown by an increase in fluorescence over that induces by agonist alone. Similarly, to test for nAChR α7 agonist activity, test compounds along with an α7 modulator are applied simultaneously to all wells. Receptor activation is measured by calcium influx into cells which is quantified by the increase in fluorescence intensity of each well. An agonist effect is determined by the increase in fluorescence over that induced by a modulator alone.

Cell-cultured neurons may be prepared as follows. Eighteen day old Sprague-Dawley rat fetuses (E-18) are aseptically removed from a pregnant female, sacrificed, the frontal cortices of the brains removed, the meninges stripped, and the cleaned cortex placed into cold HBSS. If hippocampal tissue is desired, the hippocampus is dissected away from the cortex and then placed into cold HBSS. The tissues are mechanically dispersed, washed once in HBSS (200 g for 30 min in 4° C.) suspended in a Sato's medium supplemented with glutamine, antibiotics, potassium chloride, insulin, transferrin, selenium, and 5% heat-inactivated endotoxin-free fetal bovine serum (FBS) and plated into each of a 24-well plate (coated with poly-L-lysine). The wells may contain glass cover slips which are also coated with PLL. The plates are incubated at 37° C. in a CO₂ incubator. After 24 hours the medium is removed, fresh medium added, and the cells allowed to grow for at least another 11 days, feeding when necessary.

Compounds described herein cause a 2-fold increase (100% potentiation) of baseline current as measured baseline to peak at low concentration of acetylcholine (30 μM), indicating that they are expected to have useful therapeutic activity. Compounds described herein also increase the flux of Ca⁺⁺ when applied in the Ca2+ flux-imaging assay. Any increase of Ca⁺⁺ flux, caused by a compound described herein, compared to the Ca⁺⁺ flux caused by an agonist alone (as measured in Fluorescence Intensity Units) indicates that they are expected to have useful therapeutic activity.

### General Chemical Procedures

The invention is illustrated by, but not limited to, examples described herein in which descriptions, where applicable and unless otherwise stated, the following terms, abbreviations and conditions are used:

Commercial reagents were used without further purification.

The following abbreviations are used herein: aq., aqueous; atm, atmospheric pressure; BOC, 1,1-dimethylethoxycarbonyl; DCM, dichloromethane; DMF, N,N-dimethylformamide; DMSO, dimethyl sulfoxide; EtOH, ethanol; Et₂O, diethyl ether; EtOAc, ethyl acetate; h, hour(s); HATU, 2-(7-aza-1H-benzotriazole-1-yl)-1,1,3,3-tetramethyluroniuim hexafluorophosphate; HPLC, high pressure liquid chromatography; HOBT, 1-hydroxybenzotriazole; MeOH, methanol; min, minutes; MS, mass spectrum; NMR, nuclear magnetic resonance; psi, pounds per square inch; RT, room temperature; sat., saturated; TEA, triethylamine; TFA, trifluoroacetic acid; THF, tetrahydrofuran.

Temperatures are given in degrees Celsius (° C.); unless otherwise stated, operations were carried out at room or ambient temperature (18-25° C.).

Organic solutions were dried over anhydrous sodium or magnesium sulfate; evaporation of solvent was carried out using a rotary evaporator under reduced pressure (4.5-30 mm Hg) with a bath temperature of up to 60° C.

Chromatography means flash column chromatography on silica gel unless otherwise noted; solvent mixture compositions are given as volume percentages or volume ratios.

When given, NMR data is in the form of delta values for major diagnostic protons (given in parts per million (ppm) relative to tetramethylsilane as an internal standard) determined at 300 MHz.

Melting points are uncorrected.

Mass spectra were recorded using either a Hewlett Packard 5988A or a MicroMass Quattro-1 Mass Spectrometer and are reported as m/z for the parent molecular ion. Room temperature refers to 20-25° C.

Reactions described herein, unless otherwise noted, are usually conducted at a pressure of about one to about three atmospheres, preferably at ambient pressure (about one atmosphere).

Unless otherwise stated, the reactions are conducted under an inert atmosphere, preferably under a nitrogen atmosphere.

The compounds described herein and intermediates may be isolated from their reaction mixtures by standard techniques.

As used herein, unless otherwise indicated, “C₁₋₆alkyl” includes methyl, ethyl, n-propyl, n-butyl, i-propyl, i-butyl, t-butyl, s-butyl, and the like, and C₃₋₈alkyl moieties may be straight-chained, branched or cyclic, for example cyclopropyl or cyclobutyl.

As used herein, unless otherwise indicated, “C₂₋₄alkenyl” includes but is not limited to 1-propenyl, 2-propenyl, 1-butenyl, 2-butenyl and 3-butenyl.

As used herein, unless otherwise indicated, “C₂₋₄alkynyl” includes but is not limited to ethynyl, 1-propynyl, 2-propynyl, 1-butynyl, 2-butynyl and 3-butynyl.

As used herein “halogen” means fluoride, chloride, bromide, or iodide.

### EXAMPLES

Compounds described herein may be made generally by the processes illustrated herein. In all processes described herein, where necessary, hydroxy, amino or other reactive groups may be protected using protecting groups as will be understood by those of skill in the art.

Compounds of Formula I may be prepared generally as illustrated in Scheme I.

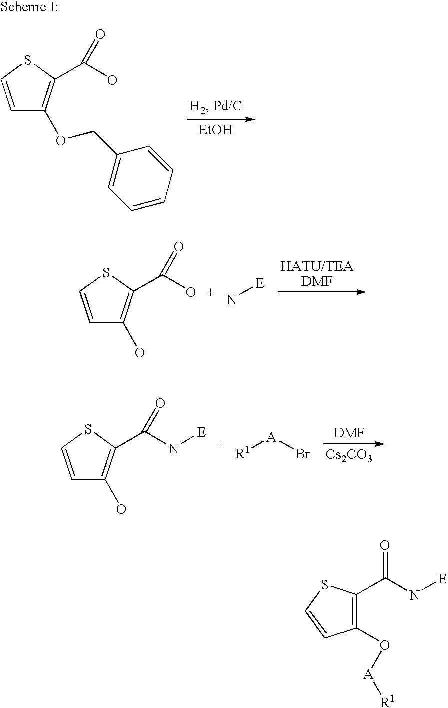

Thus, 3-benzyloxy-thiophene-2-carboxylic acid may be reduced with hydrogen with palladium on carbon in ethanol solution. The resulting 3-hydroxy-thiophene-2-carboxylic acid may be reacted in dimethyl formamide with an amine in the presence of HATU/triethylamine to form a N-substituted 3-hydroxy-thiophene-2-carboxamide. The carboxamide may then be reacted with an alkyl bromide or a substituted alkyl bromide in dimethylformamide in the presence of cesium carbonate to form a compound of Formula I.

Compounds in accord with Formula I wherein E is of Formula II may be made as illustrated in Scheme I and as more particularly illustrated in Scheme II.

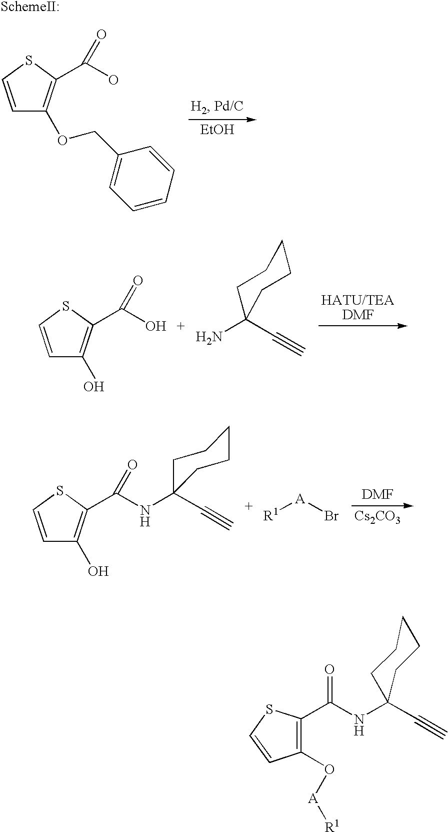

Compounds of Examples 1 through 30 were prepared according to the general procedure described in Scheme II.

Compounds in accord with Formula I wherein E is of Formula III may be made as illustrated in Scheme I and as more particularly illustrated in Scheme III.

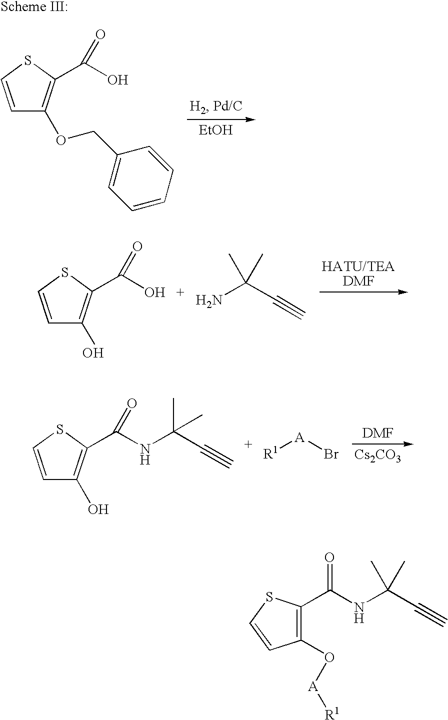

Compounds of Examples 31 through 50 were prepared according to the general procedure described in Scheme III.

### Example 1

### 3-(Benzyloxy)-N-(1-ethynylcyclohexyl)thiophene-2-carboxamide

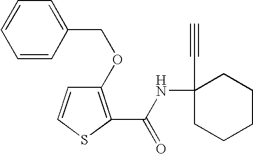

To a solution of N-(1-ethynylcyclohexyl)-3-hydroxythiophene-2 carboxamide (1b) (40 mg) in DMF (3 mL) was added Cs₂CO₃ (105 mg), benzyl bromide (27.5 mg). The reaction mixture was stirred 12 h at room temperature, solids were removed by filtration and concentrated in vacuo. The product was purified by prep-HPLC to afford the title compound (43 mg, 80%) as an off-white solid. ¹H NMR (300 MHz, CDCl₃) δ 1.47-1.72 (m, 10H), 2.35 (s, 1H), 5.16 (s, 2H), 6.22 (s, 1H), 6.89 (d, 1H), 7.25 (d, 1H), 7.32-7.40 (m, 5H). MS APCI, m/z=340 (M+1). LC/MS: 2.89 min.

### 3-hydroxythiophene-2-carboxylic acid (1a)

A mixture of 3-(benzyloxy)thiophene-2-carboxylic acid (1.0 g) and 10% palladium on carbon (1.8 g) in ethanol (150 mL) and HCl (2 N, 4.5 mL) was hydrogenated at 45 psi H₂ for 2 h. The reaction mixture was filtered through a thick layer of diatomaceous earth and concentrated in vacuo to afford the title compound (600 mg, 98%) as an off-white solid. ¹H NMR (300 MHz, CDCl₃) δ 6.78 (d, 1H), 7.49 (d, 1H). MS APCI, m/z=145 (M+1). LC/MS: 1.15 min.

### N-(1-ethynylcyclohexyl)-3-hydroxythiophene-2-carboxamide (1b)

To a solution of 3-hydroxythiophene-2-carboxylic acid (1a) (540 mg) in DMF (30 mL) at room temperature under nitrogen was added HATU (1.43 g), triethylamine (533 μL) and 1-ethynylcyclohexyl amine (462 mg). The reaction mixture was stirred 12 h at RT, concentrated in vacuo. The product was purified by flash chromatography (15% ethyl acetate/hexane) to afford the title compound (560 mg, 60%) as an off-white solid. ¹H NMR (300 MHz, CDCl₃) δ 1.41-1.70 (m, 10H), 2.45 (s, 1H), 6.75 (d, 1H), 7.50 (d, 1H). MS APCI, m/z=250 (M+1). LC/MS: 2.25 min.

### Example 2

### 3-[2-(Benzyloxy)ethoxy]-N-(1-ethynylcyclohexyl)thiophene-2-carboxamide

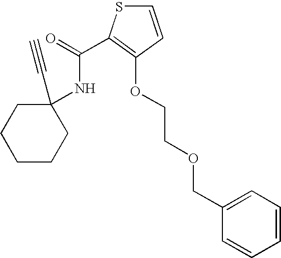

Using a procedure similar to that described in Example 1, except using [2-bromoethoxy)methyl]benzene (35 mg), the title compound was obtained as an off-white solid (25 mg, 41%). ¹H NMR (300 MHz, CDCl₃) δ 1.46-1.72 (m, 10H), 2.45 (s, 1H), 3.66 (t, 2H), 4.59 (s, 2H), 4.65 (t, 2H), 6.60 (d, 1H), 7.24 (d, 1H), 7.25-7.34 (m, 5H). MS APCI, m/z=384 (M+1).

### Example 3

### 3-(Cyclopropylmethoxy)-N-(1-ethynylcyclohexyl)thiophene-2-carboxamide

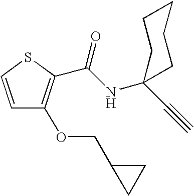

Using a procedure similar to that described in Example 1, except using cyclopropylmethyl bromide (22 mg), the title compound was obtained as an off-white solid (24 mg, 70%). ¹H NMR (300 MHz, CDCl₃) δ 0.31 (m, 2H), 0.60 (m, 2H), 1.25 (m, 1H), 1.46-1.72 (m, 10H), 2.45 (s, 1H), 4.13 (d, 2H), 6.46 (d, 1H), 7.33 (d, 1H). MS APCI, m/z=304 (M+1).

### Example 4

### N-(1-Ethynylcyclohexyl)-3(pyridin-4-ylmethoxy)thiophene-2-carboxamide

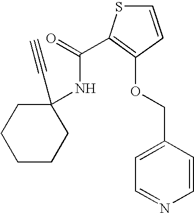

Using a procedure similar to that described in Example 1, except using 4-(bromomethyl)pyridine (28 mg), the title compound was obtained as an off-white solid (23 mg, 43%). ¹H NMR (300 MHz, CDCl₃) δ 1.48-1.72 (m, 10H), 2.45 (s, 1H), 5.33 (s, 2H), 6.54 (d, 1H), 7.15 (d, 1H), 7.46 (d, 2H), 8.61 (d, 2H). MS APCI, m/z=341 (M+1).

### Example 5

### 3-[(3-Cyclobenzyl)oxy]-N-(1-ethynylcyclohexyl)thiophene-2-carboxamide

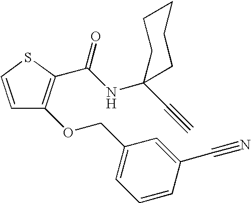

Using a procedure similar to that described in Example 1 except using 3-(bromomethyl)benzonitrile (32 mg), the title compound was obtained as an off-white solid (32 mg, 55%). ¹H NMR (300 MHz, CDCl₃) δ 1.49-1.73 (m, 10H), 2.45 (s, 1H), 5.34 (s, 2H), 6.54 (d, 1H), 7.15 (d, 1H), 7.42-7.66 (m, 4H). MS APCI, m/z=365 (M+1).

### Example 6

### N-(1-Ethynylcyclohexyl)-3-(2-oxo-2-pyridin-2-ylethoxy)thiophene-2-carboxamide

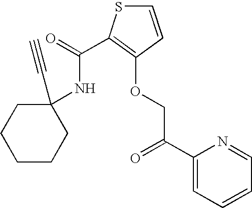

Using a procedure similar to that described in Example 1 except using 2-bromo-1-pyridin-2-ylethanone (32 mg), the title compound was obtained as an off-white solid (33 mg, 56%). ¹H NMR (300 MHz, CDCl₃) δ 1.49-1.72 (m, 10H), 2.46 (s, 1H), 5.70 (s, 2H), 6.46 (d, 1H), 7.17 (d, 1H), 7.44-8.33 (m, 4H). MS APCI, m/z=369 (M+1).

### Example 7

### N-(1-Ethynylcyclohexyl)-3-[(5-methylisoxazol-3-yl)methoxy]thiophene-2-carboxamide

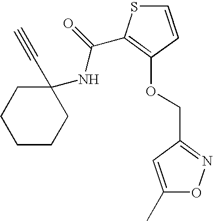

Using a procedure similar to that described in Example 1 except using 3-(bromomethyl)-5-methylisoxazole (28 mg), the title compound was obtained as an off-white solid (29 mg, 53%). ¹H NMR (300 MHz, CDCl₃) δ 1.48-1.72 (m, 10H), 2.26 (s, 3H), 2.45 (s, 1H), 5.40 (s, 2H), 6.37 (s, 1H), 6.53 (d, 1H), 7.15 (d, 1H). MS APCI, m/z=345 (M+1).

### Example 8

### 3-(Cyanomethoxy)-N-(1-ethynylcyclohexyl)thiophene-2-carboxamide

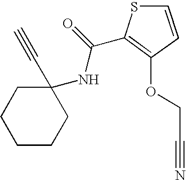

Using a procedure similar to that described in Example 1 except using bromoacetonitrile (20 mg), the title compound was obtained as an off-white solid (15 mg, 33%). ¹H NMR (300 MHz, CDCl₃) δ 1.49-1.72 (m, 10H), 2.45 (s, 1), 5.13 (s, 2H), 6.78 (d, 1H), 7.37 (d, 1H). MS APCI, m/z=289 (M+1).

### Example 9

### 3-(2,1,3-Benzoxadiazol-5-ylmethoxy)-N-(1-ethynylcyclohexyl)thiophene-2-carboxamide

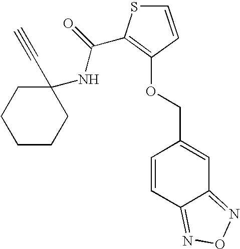

Using a procedure similar to that described in Example 1 except using 5-(bromomethyl)-2,1,3-benzoxadiazole (34 mg), the title compound was obtained as an off-white solid (10 mg, 17%). ¹H NMR (300 MHz, CDCl₃) δ 1.41-1.72 (m, 10H), 2.43 (s, 1H), 5.45 (s, 2H), 6.54 (d, 1H), 7.15 (d, 1H), 7.44-7.70 (m, 3H). MS APCI, m/z=382 (M+1).

### Example 10

### N-(1-Ethynylcyclohexyl)-3-(2-oxo-2-phenylethoxy)thiophene-2-carboxamide

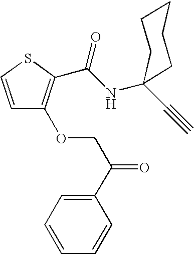

Using a procedure similar to that described in Example 1 except using 2-bromo-1-phenylethanone (32 mg), the title compound was obtained as an off-white solid (11 mg, 19%). ¹H NMR (300 MHz, CDCl₃) δ 1.49-1.72 (m, 10H), 2.45 (s, 1H), 5.59 (s, 2H), 6.46 (d, 1H), 7.17 (d, 1H), 7.54-8.05 (m, 5H). MS APCI, m/z=368 (M+1).

### Example 11

### N-(1-Ethynylcyclohexyl)-3-[2-(4-methoxyphenyl)-2-oxoethoxy]thiophene-2-carboxamide

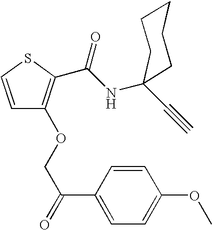

Using a procedure similar to that described in Example 1 except using 2-bromo-1-(4-methoxyphenyl)ethanone (37 mg), the title compound was obtained as an off-white solid (25 mg, 39%). ¹H NMR (300 MHz, CDCl₃) δ 1.47-1.72 (m, 10H), 2.45 (s, 1H), 3.86 (s, 3H), 5.59 (s, 2H), 6.46 (d, 1H), 6.87 (d, 2H), 7.17 (d, 1H), 8.03 (d, 2H). MS APCI, m/z=398 (M+1).

### Example 12

### 3[2-(1-Benzofuran-2-yl)-2-oxoethoxy]-N-(1-ethynyleyclohexyl thiophene-2-carboxamide

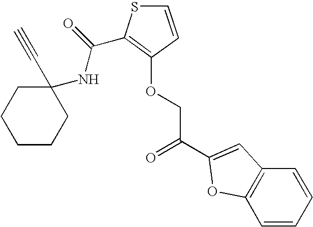

Using a procedure similar to that described in Example 1 except using 1-(1-benzofuran-2-yl)-2-bromoethanone (38 mg), the title compound was obtained as an off-white solid (10 mg, 15%). ¹H NMR (300 MHz, CDCl₃) δ 1.49-1.72 (m, 10H), 2.45 (s, 1H), 5.46 (s, 2H), 6.46 (d, 1H), 6.99 (s, 1H), 7.17 (d, 1H), 7.29-7.61 (m, 4H). MS APCI, m/z=408 (M+1).

### Example 13

### N-(1-Ethynylcyclohexyl)-3-[2-(5-methyl-1-phenyl-1H-pyrazol-4-yl)-2-oxoethoxy]thiophene-2-carboxamide

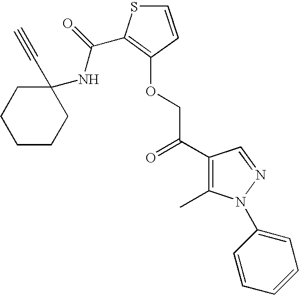

Using a procedure similar to that described in Example 1 except using 2-bromo-1-(5-methyl-1-phenyl-1H-pyrazol-4-yl)ethanone (45 mg), the title compound was obtained as an off-white solid (37 mg, 52%). ¹H NMR (300 MHz, CDCl₃) δ 1.44-1.72 (m, 10H), 2.19 (s, 3H), 2.45 (s, 1H), 5.48 (s, 2H), 6.46 (d, 1H), 7.17 (d, 1H), 7.51 (s, 1H), 7.61-7.66 (m, 5H). MS APCI, m/z=448 (M+1).

### Example 14

### N-(1-Ethynylcyclohexyl)-3-[2-(4-methylphenyl)-2-oxoethoxy]thiophene-2-carboxamide

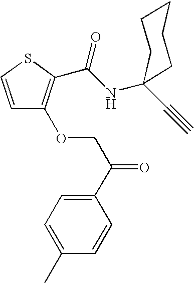

Using a procedure similar to that described in Example 1 except using 2-bromo-1-(4-methylphenyl)ethanone (34 mg), the title compound was obtained as an off-white solid (20 mg, 33%). ¹H NMR (300 MHz, CDCl₃) δ 1.35-1.96 (m, 10H), 2.39 (s, 3H), 2.45 (s, 1H), 5.43 (s, 2H), 6.82 (d, 1H), 7.23 (d, 1H), 7.35 (d, 2H), 7.83 (d, 2H). MS APCI, m/z=382 (M+1).

### Example 15

### 3-[2-(4-Chlorophenyl)-2-oxoethoxy]-N-(1-ethynylcyclohexyl)thiophene-2-carboxamide

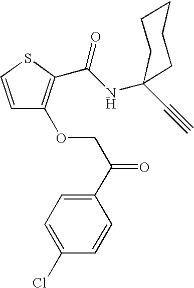

Using a procedure similar to that described in Example 1 except using 2-bromo-1-(4-chlorophenyl)ethanone (37 mg), the title compound was obtained as an off-white solid (10 mg, 16%). ¹H NMR (300 MHz, CDCl₃) δ 1.49-1.72 (m, 10H), 2.45 (s, 1H), 5.59 (s, 2H), 6.45 (d, 1H), 7.18 (d, 1H), 7.46 (d, 2H), 7.89 (d, 2H). MS APCI, m/z=402 (M+1).

### Example 16

### 3-(1,3-Dioxolan-2-ylmethoxy)-N-(1-ethynylcyclohexyl)thiophene-2-carboxamide

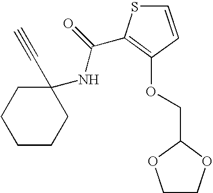

Using a procedure similar to that described in Example 1 except using 2-(bromomethyl)-1,3-dioxolane (27 mg), the title compound was obtained as an off-white solid (5 mg, 9.4%). ¹H NMR (300 MHz, CDCl₃) δ 1.45-1.70 (m, 10H), 2.43 (s, 1H), 3.93 (t, 2H), 3.99 (t, 2H), 4.41 (s, 2H), 5.32 (t, 1H), 6.54 (d, 1H), 7.21 (d, 2H). MS APCI, m/z=336 (M+1).

### Example 17

### N-(1-Ethynylcyclohexyl)-3-(2-oxo-2-pyridin-3-ylethoxy)thiophene-2-carboxamide

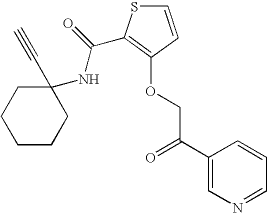

Using a procedure similar to that described in Example 1 except using 2-bromo-1-pyridin-3-ylethanone (32 mg), the title compound was obtained as an off-white solid (11 mg, 19%). ¹H NMR (300 MHz, CDCl₃) δ 1.49-1.72 (m, 100H), 2.45 (s, 1H), 5.70 (s, 2H), 6.54 (d, 1H), 7.17 (d, 1H), 7.46-8.33 (m, 4H). MS APCI, m/z=369 (M+1).

### Example 18

### 3-Ethoxy-N-(1-ethynylcyclohexyl)thiophene-2-carboxamide

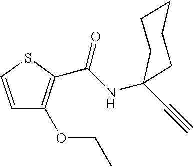

Using a procedure similar to that described in Example 1 except using ethyl bromide (18 mg), the title compound was obtained as an off-white solid (15 mg, 34%). ¹H NMR (300 MHz, CDCl₃) δ 1.40-1.71 (m, 10H), 1.43 (t, 3H), 2.35 (s, 1H), 4.39 (q, 2H), 6.46 (d, 1H), 7.18 (d, 1H). MS APCI, m/z=278 (M+1).

### Example 19

### N-(1-Ethynylcyclohexyl)-3-(2-phenylethoxy)thiophene-2-carboxamide

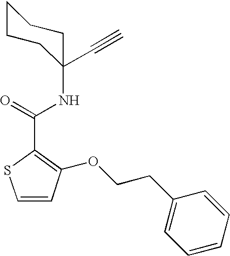

Using a procedure similar to that described in Example 1 except using (2-bromoethyl)benzene (30 mg), the title compound was obtained as an off-white solid (40 mg, 71%). ¹H NMR (300 MHz, CDCl₃) δ 1.32-2.05 (m, 10H), 2.47 (s, 1H), 3.16 (t, 2H), 4.43 (t, 2H), 6.72 (d, 1H), 7.15-7.20 (m, 5H), 7.20 (d, 1H). MS APCI, m/z=354 (M+1). LC/MS=2.88 min.

### Example 20

### N-(1-Ethynyleyclohexyl)-3-[(4-fluorobenzyl)oxy]thiophene-2-carboxamide

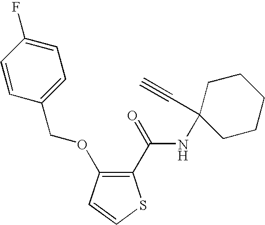

Using a procedure similar to that described in Example 1 except using 4-fluorobenzyl bromide (30.2 mg), the title compound was obtained as an off-white solid (40 mg, 70%). ¹H NMR (300 MHz, CDCl₃) δ 1.27-1.95 (m, 10H), 2.35 (s, 1H), 5.20 (s, 2H), 6.88 (d, 1H), 7.15 (d, 1H), 7.24 (d, 2H), 7.58 (d, 2H). MS APCI, m/z=358 (M+1). LC/MS=2.89 min.

### Example 21

### N-(1-Ethynylcyclohexyl)-3-[2-(1H-indol-3-yl)ethoxy]thiophene-2-carboxamide

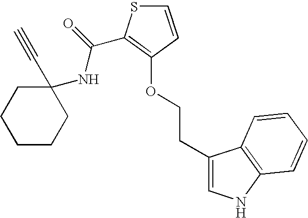

Using a procedure similar to that described in Example 1 except using 3-(2-bromoethyl)-1H-indole (36 mg), the title compound was obtained as an off-white solid (10 mg, 16%). ¹H NMR (300 MHz, CDCl₃) δ 1.46-1.73 (m, 10H), 2.45 (s, 1H), 3.17 (t, 2H), 4.53 (t, 2H), 6.42 (d, 1H), 6.68 (d, 1H), 7.23 (d, 1H), 7.24-7.56 (m, 4H). MS APCI, m/z=393 (M+1).

### Example 22

### N-(1-Ethynylcyclohexyl)-3-[2-(2-methoxyphenyl)-2-oxoethoxy]thiophene-2-carboxamide

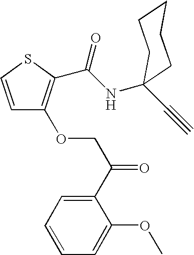

Using a procedure similar to that described in Example 1 except using 2-bromo-1-(2-methoxyphenyl)ethanone (37 mg), the title compound was obtained as an off-white solid (26 mg, 41%). ¹H NMR (300 MHz, CDCl₃) δ 1.49-1.72 (m, 10H), 2.45 (s, 1H), 3.79 (s, 3H), 5.65 (s, 2H), 6.46 (d, 1H); 7.17 (d, 1H), 7.20-7.60 (m, 4H). MS APCI, m/z=398 (M+1).

### Example 23

### N-(1-Ethynylcyclohexyl)-3-(3-methylbutoxy)thiophene-2-carboxamide

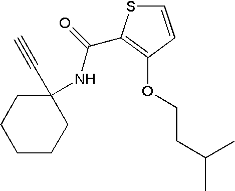

Using a procedure similar to that described in Example 1 except using 1-bromo-3-methylbutane (24 mg), the title compound was obtained as an off-white solid (25 mg, 49%). ¹H NMR (300 MHz, CDCl₃) δ 0.97 (d, 6H), 1.40-1.75 (m, 10H), 1.52 (m, 2H), 2.50 (s, 1H), 4.20 (t, 2H), 6.50 (d, 1H), 7.21 (d, 1H). MS APCI, m/z=320 (M+1).

### Example 24

### N-(1-Ethynylcyclohexyl)-3-(pyridin-2-ylmethoxy)thiophene-2-carboxamide

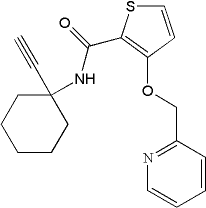

Using a procedure similar to that described in Example 1 except using 2-(bromomethyl)pyridine (28 mg), the title compound was obtained as an off-white solid (32 mg, 59%). ¹H NMR (300 MHz, CDCl₃) δ 1.48-1.72 (m, 10H), 2.45 (s, 1H), 5.52 (s, 2H), 6.54 (d, 1H), 7.15 (d, 1H), 7.24-8.23 (m, 4H). MS APCI, m/z=341 (M+1).

### Example 25

### 3-(4-Cyanobutoxy)-N-(1-ethynylcyclohexyl)thiophene-2-carboxamide

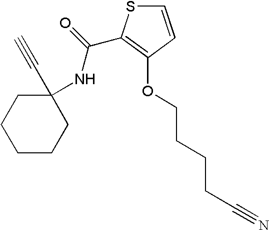

Using a procedure similar to that described in Example 1 except using 5-bromopentanenitrile (26 mg), the title compound was obtained as an off-white solid (34 mg, 64%). ¹H NMR (300 MHz, CDCl₃) δ 1.48-1.72 (m, 10H), 1.99 (m, 2H), 2.25 (t, 2H), 2.45 (s, 1H), 4.45 (t, 2H), 6.48 (d, 1H), 7.23 (d, 1H). MS APCI, m/z=331 (M+1).

### Example 26

### N-(1-Ethynylcyclohexyl)-3-[(3-methoxybenzyl)oxy]thiophene-2-carboxamide

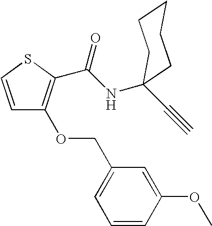

Using a procedure similar to that described in Example 1 except using 1-(bromomethyl)-3-methoxybenzene (32 mg), the title compound was obtained as an off-white solid (33 mg, 56%). ¹H NMR (300 MHz, CDCl₃) δ 1.48-1.72 (m, 10H), 2.45 (s, 1H), 3.80 (s, 3H), 5.41 (s, 2H), 6.54 (d, 1H), 6.81 (m, 1H), 7.03 (s, 1H), 7.15 (d, 1H), 7.18 (m, 1H), 7.20 (m, 1H). MS APCI, m/z=370 (M+1).

### Example 27

### N-(1-Ethynylcyclohexyl)-3-(1-phenylethoxy)thiophene-2-carboxamide

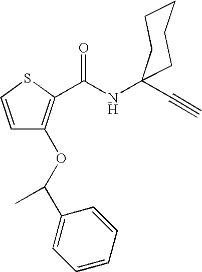

Using a procedure similar to that described in Example 1 except using 1-(bromoethyl)benzene (30 mg), the title compound was obtained as an off-white solid (39 mg, 70%). ¹H NMR (300 MHz, CDCl₃) δ 1.47-1.71 (m, 10H), 1.62 (d, 3H), 2.45 (s, 1H), 5.43 (d, 1H), 6.47 (d, 1H), 7.14 (d, 1H), 7.15-7.48 (m, 5H). MS APCI, m/z=354 (M+1).

### Example 28

### N-(1-Ethynylcyclohexyl)-3-(2-methoxyethoxy)thiophene-2-carboxamide

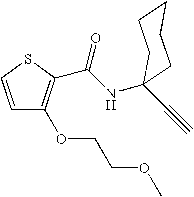

Using a procedure similar to that described in Example 1 except using 1-bromo-2-methoxyethane (22 mg), the title compound was obtained as an off-white solid (34 mg, 70%). ¹H NMR (300 MHz, CDCl₃) δ 1.45-1.81 (m, 10H), 2.45 (s, 1H), 3.37 (s, 3H), 3.80 (t, 2H), 4.64 (t, 2H), 6.60 (d, 1H), 7.24 (d, 1H). MS APCI, m/z=308 (M+1).

### Example 29

### 3-[(4-Cyanobenzyl)oxy]-N-(1-ethynyleyclohexyl)thiophene-2-carboxamide

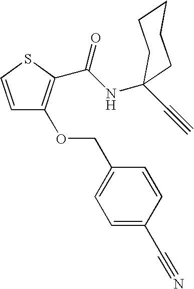

Using a procedure similar to that described in Example 1 except using 4-(bromomethyl)benzonitrile (32 mg), the title compound was obtained as an off-white solid (35 mg, 60%). ¹H NMR (300 MHz, CDCl₃) δ 1.49-1.74 (m, 10H), 2.45 (s, 1H), 5.36 (s, 2H), 6.53 (d, 1H), 7.14 (d, 1H), 7.62 (d, 2H), 7.65 (d, 2H). MS APCI, m/z=365 (M+1).

### Example 30

### N-(1-Ethynylcyclohexyl)-3-(pyridin-3-ylmethoxy)thiophene-2-carboxamide

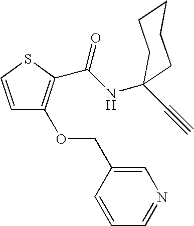

Using a procedure similar to that described in Example 1 except using 3-(bromomethyl)pyridine (28 mg), the title compound was obtained as an off-white solid (38 mg, 70%). ¹H NMR (300 MHz, CDCl₃) δ 1.48-1.72 (m, 10H), 2.45 (s, 1H), 5.43 (s, 2H), 6.60 (d, 1H), 7.11 (m, 1H), 7.15 (d, 1H), 7.66 (d, 1H), 8.34 (d, 1H), 8.8 (d, 1H). MS APCI, m/z=341 (M+1).

### Example 31

### 3-(Benzyloxy)-N-(1,1-dimethylprop-2-yn-1-yl)thiophene-2-carboxamide

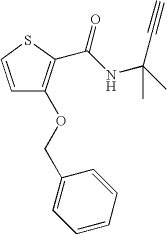

To a solution of N-(1,1-dimethylprop-2-yn-1-yl)-3-hydroxythiophene-2 carboxamide (2b) (40 mg) in DMF (3 mL) was added Cs₂CO₃ (125 mg) and benzyl bromide (33 mg). The reaction mixture was stirred 12 h at RT, filtered to remove solids and concentrated in vacuo. The product was purified by prep-HPLC to afford the title compound (45 mg, 80%) as an off-white solid. ¹H NMR (300 MHz, CDCl₃) δ 1.60 (s, 6H), 2.25 (s, 1H), 5.16 (s, 2H), 6.89 (d, 1H), 7.35 (d, 1H), 7.37-7.41 (m, 5H). MS APCI, m/z=300 (M+1). LC/MS: 2.65 min.

### N-(1,1-Dimethylprop-2-yn-1-yl)-3-hydroxythiophene-2-carboxamide (2b)

To a solution of 3-hydroxythiophene-2-carboxylic acid (1a) (645 mg) in DMF (15 mL) at RT under nitrogen was added HATU (1.87 g), triethylamine (670 μL) and 1,1-dimethylpropargyl amine (373 mg). The reaction mixture was stirred 6 h at RT, and concentrated in vacuo. The product was purified by flash chromatography (15% ethyl acetate/hexane) to afford the title compound (468 mg, 50%) as an off-white solid. ¹H NMR (300 MHz, CDCl₃) δ 1.75 (s, 6H), 2.43 (s, 1H), 6.74 (d, 1H), 7.21 (d, 1H). MS APCI, m/z=210 (M+1). LC/MS: 1.85 min.

### Example 32

### N-(1,1-Dimethylprop-2-yn-1-yl)-3-(2-phenylethoxy)thiophene-2-carboxamide

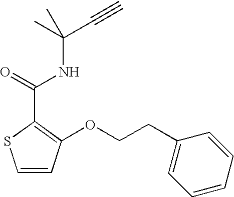

Using a procedure similar to that described in Example 31 except using (2-bromoethyl)benzene (35.2 mg), the title compound was obtained as an off-white solid (36 mg, 60%). ¹H NMR (300 MHz, CDCl₃) δ 1.62 (m, 6H), 2.35 (s, 1H), 3.20 (t, 2H), 4.41 (t, 2H), 6.82 (d, 1H), 7.24 (d, 1H), 7.21-7.77 (m, 5H). MS APCI, m/z=314 (M+1). LC/MS=2.68 min.

### Example 33

### N-(1,1-Dimethylprop-2-yn-1-yl)-3-[2-(4-methoxyphenyl)-2-oxoethoxy]thiophene-2-carboxamide

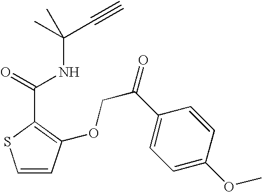

Using a procedure similar to that described in Example 31 except using 2-bromo-1-(4-methoxyphenyl)ethanone (44 mg), the title compound was obtained as an off-white solid (54 mg, 80%). ¹H NMR (300 MHz, CDCl₃) δ 1.78 (s, 6H), 2.35 (s, 1H), 3.90 (s, 3H), 5.35 (s, 2H), 6.82 (d, 1H), 6.98 (d, 2H), 7.37 (d, 1H), 7.94 (d, 2H). MS APCI, m/z=358 (M+1). LC/MS: 2.65 min.

### Example 34

### N-(1,1-Dimethylprop-2-yn-1-yl)-3-ethoxythiophene-2-carboxamide

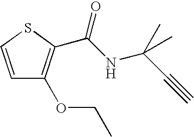

Using a procedure similar to that described in Example 31 except using ethyl bromide (21 mg), the title compound was obtained as an off-white solid (34 mg, 76%). ¹H NMR (300 MHz, CDCl₃) δ 1.48 (t, 3H), 1.74 (s, 6H), 2.35 (s, 1H), 4.39 (q, 2H), 6.81 (d, 1H), 7.45 (d, 1H). MS APCI, m/z=238 (M+1). LC/MS: 2.30 min.

### Example 35

### 3-(Cyclopropylmethoxy)-N-(1,1-dimethylprop-2-yn-1-yl)thiophene-2-carboxamide

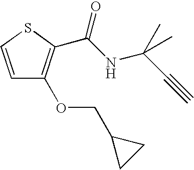

Using a procedure similar to that described in Example 31 except using (bromomethyl)cyclopropane (26 mg), the title compound was obtained as an off-white solid (38 mg, 76%). ¹H NMR (300 MHz, CDCl₃) δ 0.41 (m, 2H), 0.67 (m, 2H), 1.30 (m, 1H), 1.75 (s, 6H), 2.35 (s, 1H), 3.99 (d, 2H), 6.77 (d, 1H), 7.34 (d, 1H). MS APCI, m/z=264 (M+1). LC/MS: 2.50 min.

### Example 36

### 3-[(3-Cyanobenzyl)oxy]-N-(1,1-dimethylprop-2-yn-1-yl)thiophene-2-carboxamide

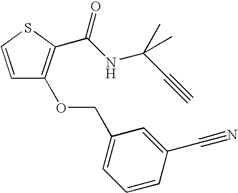

Using a procedure similar to that described in Example 31 except using 3-(bromomethyl)benzonitrile (37 mg), the title compound was obtained as an off-white solid (50 mg, 81%). ¹H NMR (300 MHz, CDCl₃) δ 1.66 (s, 6H), 2.33 (s, 1H), 5.21 (s, 2H), 6.86 (d, 1H), 7.40 (d, 1H), 7.53-7.76 (m, 4H). MS APCI, m/z=325 (M+1). LC/MS: 2.50 min.

### Example 37

### N-(1,1-Dimethylprop-2-yn-1-yl)-3-(2-oxo-phenylethoxy)thiophene-2-carboxamide

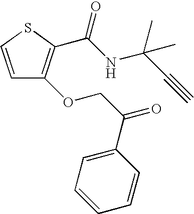

Using a procedure similar to that described in Example 31 except using 2-bromo-1-phenylethanone (38 mg), the title compound was obtained as an off-white solid (49 mg, 79%). ¹H NMR (300 MHz, CDCl₃) δ 1.78 (s, 6H), 2.36 (s, 1H), 5.41 (s, 2H), 6.83 (d, 1H), 7.39 (d, 1H), 7.52-7.96 (m, 5H). MS APCI, m/z=328 (M+1). LC/MS: 2.54 min.

### Example 38

### N-(1,1-Dimethylprop-2-yn-1-yl)-3-(2-(2-methoxyphenyl)-2-oxoethoxy]thiophene-2-carboxamide

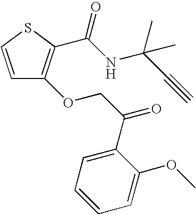

Using a procedure similar to that described in Example 31 except using 2-bromo-1-(2-methoxyphenyl)ethanone (44 mg), the title compound was obtained as an off-white solid (53 mg, 78%). ¹H NMR (300 MHz, CDCl₃) δ 1.79 (s, 6H), 2.36 (s, 1H), 3.97 (s, 3H), 5.31 (s, 2H), 6.77 (d, 1H), 7.01 (m, 2H), 7.35 (d, 1H), 7.56-8.04 (m, 2H). MS APCI, m/z=358 (M+1). LC/MS: 2.62 min.

### Example 39

### N-(1,1-Dimethylprop-2-yn-1-yl)-3-(2-(4-methylphenyl)-2-oxoethoxy]thiophene-2-carboxamide

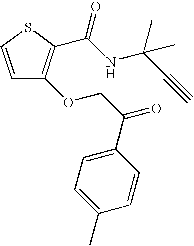

Using a procedure similar to that described in Example 31 except using 2-bromo-1-(4-methylphenyl)ethanone (41 mg), the title compound was obtained as an off-white solid (46 mg, 71%). ¹H NMR (300 MHz, CDCl₃) δ 1.77 (s, 6H), 2.35 (s, 1H), 2.45 (s, 3H), 5.38 (s, 2H), 6.81 (d, 1H), 7.31 (d, 1H), 7.37 (d, 2H), 7.84 (d, 2H). MS APCI, m/z=342 (M+1). LC/MS: 2.66 min.

### Example 40

### N-(1,1-Dimethylprop-2-yn-1-yl)-3-(pyridin-4-ylmethoxy)thiophene-2-carboxamide

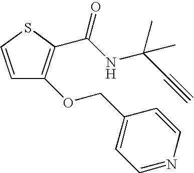

Using a procedure similar to that described in Example 31 except using 4-(bromomethyl)pyridine (33 mg), the title compound was obtained as an off-white solid (29 mg, 51%). ¹H NMR (300 MHz, CDCl₃) δ 1.69 (s, 6H), 2.32 (s, 1H), 5.23 (s, 2H), 6.83 (d, 1H), 7.28 (d, 1H), 7.39 (d, 2H), 8.69 (d, 21). MS APCI, m/z=301 (M+1). LC/MS: 1.64 mm.

### Example 41

### 3-(Cyanomethoxy)-N-(1,1-dimethylprop-2-yn-1-yl)thiophene-2-carboxamide

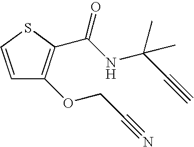

Using a procedure similar to that described in Example 31 except using bromoacetonitrile (23 mg), the title compound was obtained as an off-white solid (35 mg, 74%). ¹H NMR (300 MHz, CDCl₃) δ 1.75 (s, 6H), 2.37 (s, 1H), 4.89 (s, 2H), 6.89 (d, 1H), 7.45 (d, 1H). MS APCI, m/z=249 (M+1). LC/MS: 2.06 min.

### Example 42

### N-(1,1-Dimethylprop-2-yn-1-yl)-3-(1,3-dioxolan-2-ylmethoxy)thiophene-2-carboxamide

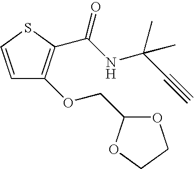

Using a procedure similar to that described in Example 31 except using 2-(bromomethyl)-1,3-dioxolane (32 mg), the title compound was obtained as an off-white solid (30 mg, 50%). ¹H NMR (300 MHz, CDCl₃) δ 1.73 (s, 6H), 2.35 (s, 1H), 3.95-4.03 (m, 4H), 4.18 (s, 2H), 5.23 (t, 1H), 6.82 (d, 1H), 7.35 (d, 1H). MS APCI, m/z=296 (M+1). LC/MS: 2.14 min.

### Example 43

### N-(1,1-Dimethylprop-2-yn-1-yl)-3-[2-(1H-indo-3-yl)ethoxy]thiophene-2-carboxamide

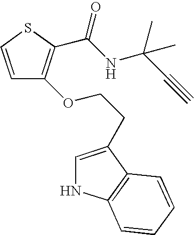

Using a procedure similar to that described in Example 31 except using 3-(2-bromoethyl)-1H-indole (43 mg), the title compound was obtained as an off-white solid (12 mg, 18%). ¹H NMR (300 MHz, CDCl₃) δ 1.55 (s, 6H), 2.27 (s, 1H), 3.03 (t, 2H), 4.49 (t, 2H), 6.83 (d, 1H), 7.10 (d, 1H), 7.14-7.60 (m, 4H), 7.36 (d, 1H). MS APCI, m/z=353 (M+1). LC/MS: 2.63 min.

### Example 44

### N-(1,1-Dimethylprop-2-yn-1-yl)-3-(2-oxo-pyridin-3-ylethoxy)thiophene-2-carboxamide

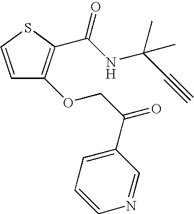

Using a procedure similar to that described in Example 31 except using 2-bromo-1-pyridin-3-ylethanone (38 mg), the title compound was obtained as an off-white solid (37 mg, 59%). ¹H NMR (300 MHz, CDCl₃) δ 1.79 (s, 6H), 2.36 (s, 1H), 5.43 (s, 2H), 6.84 (d, 1H), 7.41 (d, 1H), 7.50 (m, 1H), 8.23 (m, 1H), 8.87 (m, 1H), 9.16 (d, 1H). MS APCI, m/z=329 (M+1). LC/MS: 2.18 min.

### Example 45

### 3-(2,1,3-Benzoxadiazol-5-ylmethoxy)-N-(1,1-dimethylprop-2-yn-1-yl)thiophene-2-carboxamide

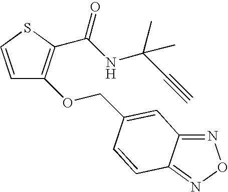

Using a procedure similar to that described in Example 31 except using 5-(bromomethyl)-2,1,3-benzoxadiazole (41 mg), the title compound was obtained as an off-white solid (47 mg, 73%). ¹H NMR (300 MHz, CDCl₃) δ 1.70 (s, 6H), 2.35 (s, 1H), 5.30 (s, 2H), 6.90 (d, 1H), 7.42 (d, 1H), 7.43-7.96 (m, 3H). MS APCI, m/z=342 (M+1). LC/MS: 2.58 min.

### Example 46

### 3-[2-(1-Benzofuran-2-yl)-2-oxoethoxy]-N-(1,1-dimethylprop-2-yn-1-yl)thiophene-2-carboxamide

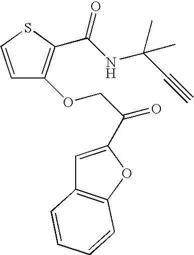

Using a procedure similar to that described in Example 31 except using 1-(1-benzofuran-2-yl)-2-bromoethanone (45 mg), the title compound was obtained as an off-white solid (36 mg, 52%). ¹H NMR (300 MHz, CDCl₃) δ 1.81 (s, 6H), 2.36 (s, 1H), 5.42 (s, 2H), 6.86 (d, 1H), 7.34 (s, 1H), 7.40 (d, 1H), 7.53-7.88 (m, 4H). MS APCI, m/z=368 (M+1). LC/MS: 2.63 min.

### Example 47

### N-(1,1-Dimethylprop-2-yn-1-yl)-3-(2-oxo-2-pyridin-2-ylethoxy)thiophene-2-carboxamide

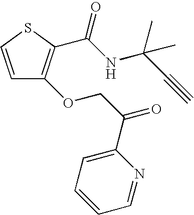

Using a procedure similar to that described in Example 31 except using 2-bromopyridin-2-ylethanone (38 mg), the title compound was obtained as an off-white solid (37 mg, 59%). ¹H NMR (300 MHz, CDCl₃) δ 1.84 (s, 6H), 2.38 (s, 1H), 5.72 (s, 2H), 6.89 (d, 1H), 7.37 (s, 1H), 7.56 (m, 1H), 7.89 (m, 1H), 8.10 (d, 1H), 8.68 (d, 1H). MS APCI, m/z=329 (M+1). LC/MS: 2.38 min.

### Example 48

### N-(1,1-Dimethylprop-2-yn-1-yl)-3-[(5-methylisoxazol-3-yl)methoxy]thiophene-2-carboxamide

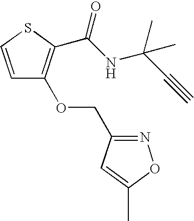

Using a procedure similar to that described in Example 31 except using 3-(bromomethyl)-5-methylisoxazole (34 mg), the title compound was obtained as an off-white solid (42 mg, 73%). ¹H NMR (300 MHz, CDCl₃) δ 1.71 (s, 6H), 2.33 (s, 1H), 2.45 (s, 3H), 5.24 (s, 2H), 6.09 (s, 1H), 6.90 (d, 1H), 7.37 (d, 1H). MS APCI, m/z=305 (M+1). LC/MS: 2.33 min.

### Example 49

### N-(1,1-Dimethylprop-2-yn-1-yl)-3-[(5-methyl-2-phenyl-2H-1,2,3-triazol-4-yl)methoxy]thiophene-2-carboxamide

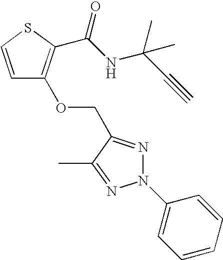

Using a procedure similar to that described in Example 31 except using 4-(bromomethyl)-5-methyl-2-phenyl-2H-1,2,3-triazole (48 mg), the title compound was obtained as an off-white solid (57 mg, 79%). ¹H NMR (300 MHz, CDCl₃) δ 1.64 (s, 6H), 2.24 (s, 1H), 2.45 (s, 3H), 5.33 (s, 2H), 7.01 (s, 1H), 7.32 (d, 1H), 7.36-8.02 (m, 5H). MS APCI, m/z=381 (M+1). LC/MS: 2.78 min.

### Example 50

### 3-[2-(Benzyloxy)ethoxy]-N-(1,1-dimethylprop-2-yn-1-yl)thiophene-2-carboxamide

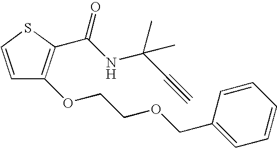

Using a procedure similar to that described in Example 31 except using [(2-bromoethoxy)methyl]benzene (41 mg), the title compound was obtained as an off-white solid (55 mg, 84%). ¹H NMR (300 MHz, CDCl₃) δ 1.67 (s, 6H), 2.32 (s, 1H), 3.81 (m, 2H), 4.30 (m, 2H), 4.60 (s, 2H), 6.80 (d, 1H), 7.30 (d, 1H), 7.31-7.52 (m, 5H). MS APCI, m/z=344 (M+1). LC/MS: 2.65 min.

1. A compound in accord with Formula I. 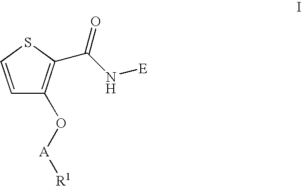
wherein:
E is selected from a moiety of formula II or III, 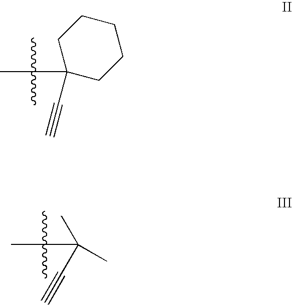
A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and
R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:
R¹ may be unsubstituted or be substituted with 1 or 2 moieties selected from CN, phenyl, halogen, —C₁₋₄alkyl and —C₁₋₄alkoxy,
or a stereoisomer, enantiomer, in vivo-hydrolysable precursor or pharmaceutically-acceptable salt thereof.

2. A compound according to claim 1, wherein: 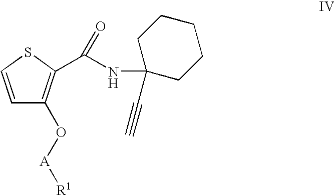
wherein:
A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and
R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:
R¹ may be unsubstituted or be substituted with 1 or 2 moieties selected from CN, phenyl, halogen, —C₁₋₄alkyl and —C₁₋₄alkoxy,
or a stereoisomer, enantiomer, in vivo-hydrolysable precursor or pharmaceutically-acceptable salt thereof.

3. A compound according to claim 1, wherein: 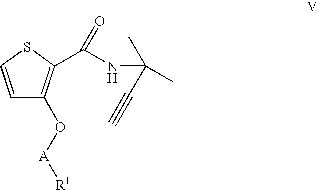
wherein:
A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and
R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:
R¹ may be unsubstituted or be substituted with 1 or 2 moieties selected from CN, phenyl, halogen, —C₁₋₄alkyl and —C₁₋₄alkoxy,
or a stereoisomer, enantiomer, in vivo-hydrolysable precursor or pharmaceutically-acceptable salt thereof.

4. A compound according to claim 1, selected from:
3-(Benzyloxy)-N-(1-ethynylcyclohexyl)thiophene-2-carboxamide;
3-[2-(Benzyloxy)ethoxy]-N-(1-ethynylcyclohexyl)thiophene-2-carboxamide;
3-(Cyclopropylmethoxy)-N-(1-ethynylcyclohexyl)thiophene-2-carboxamide;
N-(1-Ethynylcyclohexyl)-3 (pyridin-4-ylmethoxy)thiophene-2-carboxamide;
3-[(3-Cyclobenzyl)oxy]-N-(1-ethynylcyclohexyl)thiophene-2-carboxamide;
N-(1-Ethynylcyclohexyl)-3-(2-oxo-2-pyridin-2-ylethoxy)thiophene-2-carboxamide;
N-(1-Ethynylcyclohexyl)-3-[(5-methylisoxazol-3-yl)methoxy]thiophene-2-carboxamide;
3-(Cyanomethoxy)-N-(1-ethynylcyclohexyl)thiophene-2-carboxamide;
3-(2,1,3-Benzoxadiazol-5-ylmethoxy)-N-(1-ethynylcyclohexyl)thiophene-2-carboxamide;
N-(1-Ethynylcyclohexyl)-3-(2-oxo-2-phenylethoxy)thiophene-2-carboxamide;
N-(1-Ethynylcyclohexyl)-3-[2-(4-methoxyphenyl)-2-oxoethoxy]thiophene-2-carboxamide;
3[2-(1-Benzofuran-2-yl)-2-oxoethoxy]-N-(1-ethynylcyclohexyl thiophene-2-carboxamide;
N-(1-Ethynylcyclohexyl)-3-[2-(5-methyl-1-phenyl-1H-pyrazol-4-yl)-2-oxoethoxy]thiophene-2-carboxamide;
N-(1-Ethynylcyclohexyl)-3-[2-(4-methylphenyl)-2-oxoethoxy]thiophene-2-carboxamide;
3-[2-(4-Chlorophenyl)-2-oxoethoxy]-N-(1-ethynylcyclohexyl)thiophene-2-carboxamide;
3-(1,3-Dioxolan-2-ylmethoxy)-N-(1-ethynylcyclohexyl)thiophene-2-carboxamide;
N-(1-Ethynylcyclohexyl)-3-(2-oxo-2-pyridin-3-ylethoxy)thiophene-2-carboxamide;
3-Ethoxy-N-(1-ethynylcyclohexyl)thiophene-2-carboxamide;
N-(1-Ethynylcyclohexyl)-3-(2-phenylethoxy)thiophene-2-carboxamide;
N-(1-Ethynylcyclohexyl)-3-[(4-fluorobenzyl)oxy]thiophene-2-carboxamide;
N-(1-Ethynylcyclohexyl)-3-[2-(1H-indol-3-yl)ethoxy]thiophene-2-carboxamide;
N-(1-Ethynylcyclohexyl)-3-[2-(2-methoxyphenyl)-2-oxoethoxy]thiophene-2-carboxamide;
N-(1-Ethynylcyclohexyl)-3-(3-methylbutoxy)thiophene-2-carboxamide;
N-(1-Ethynylcyclohexyl)-3-(pyridin-2-ylmethoxy)thiophene-2-carboxamide;
3-(4-Cyanobutoxy)-N-(1-ethynylcyclohexyl)thiophene-2-carboxamide;
N-(1-Ethynylcyclohexyl)-3-[(3-methoxybenzyl)oxy]thiophene-2-carboxamide;
N-(1-Ethynylcyclohexyl)-3-(1-phenylethoxy)thiophene-2-carboxamide;
N-(1-Ethynylcyclohexyl)-3-(2-methoxyethoxy)thiophene-2-carboxamide;
3-[(4-Cyanobenzyl)oxy]-N-(1-ethynylcyclohexyl)thiophene-2-carboxamide;
N-(1-Ethynylcyclohexyl)-3-(pyridin-3-ylmethoxy)thiophene-2-carboxamide;
3-(Benzyloxy)-N-(1,1-dimethylprop-2-yn-1-yl)thiophene-2-carboxamide;
N-(1,1-Dimethylprop-2-yn-1-yl)-3-(2-phenylethoxy)thiophene-2-carboxamide;
N-(1,1-Dimethylprop-2-yn-1-yl)-3-[2-(4-methoxyphenyl)-2-oxoethoxy]thiophene-2-carboxamide;
N-(1,1-Dimethylprop-2-yn-1-yl)-3-ethoxythiophene-2-carboxamide;
3-(Cyclopropylmethoxy)-N-(1,1-dimethylprop-2-yn-1-yl)thiophene-2-carboxamide;
3-[(3-Cyanobenzyl)oxy]-N-(1,1-dimethylprop-2-yn-1-yl)thiophene-2-carboxamide;
N-(1,1-Dimethylprop-2-yn-1-yl)-3-(2-oxo-phenylethoxy)thiophene-2-carboxamide;
N-(1,1-Dimethylprop-2-yn-1-yl)-3-(2-(2-methoxyphenyl)-2-oxoethoxy]thiophene-2-carboxamide;
N-(1,1-Dimethylprop-2-yn-1-yl)-3-(2-(4-methylphenyl)-2-oxoethoxy]thiophene-2-carboxamide;
N-(1,1-Dimethylprop-2-yn-1-yl)-3-(pyridin-4-ylmethoxy)thiophene-2-carboxamide;
3-(Cyanomethoxy)-N-(1,1-dimethylprop-2-yn-1-yl)thiophene-2-carboxamide;
N-(1,1-Dimethylprop-2-yn-1-yl)-3-(1,3-dioxolan-2-ylmethoxy)thiophene-2-carboxamide;
N-(1,1-Dimethylprop-2-yn-1-yl)-3-[2-(1H-indo-3-yl)ethoxy]thiophene-2-carboxamide;
N-(1,1-Dimethylprop-2-yn-1-yl)-3-(2-oxo-pyridin-3-ylethoxy)thiophene-2-carboxamide;
3-(2,1,3-Benzoxadiazol-5-ylmethoxy)-N-(1,1-dimethylprop-2-yn-1-yl)thiophene-2-carboxamide;
3-[2-(1-Benzofuran-2-yl)-2-oxoethoxy]-N-(1,1-dimethylprop-2-yn-1-yl)thiophene-2-carboxamide;
N-(1,1-Dimethylprop-2-yn-1-yl)-3-(2-oxo-2-pyridin-2-ylethoxy)thiophene-2-carboxamide;
N-(1,1-Dimethylprop-2-yn-1-yl)-3-[(5-methylisoxazol-3-yl)methoxy]thiophene-2-carboxamide;
N-(1,1-Dimethylprop-2-yn-1-yl)-3-[(5-methyl-2-phenyl-2H-1,2,3-triazol-4-yl)methoxy]thiophene-2-carboxamide, or
3-[2-(Benzyloxy)ethoxy]-N-(1,1-dimethylprop-2-yn-1-yl)thiophene-2-carboxamide,
or a stereoisomer, enantiomer, in vivo-hydrolysable precursor or pharmaceutically-acceptable salt thereof.

5. A process for preparing a compound of Formula I, 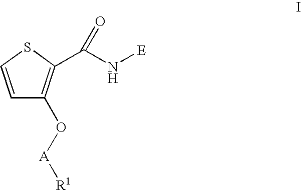
wherein:
E is selected from a moiety of formula II or III, 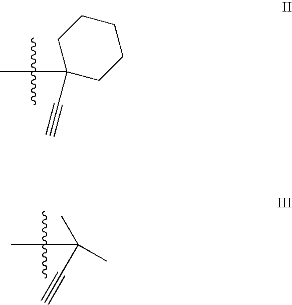
A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and
R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:
R¹ may be unsubstituted or be substituted with 1 or 2 moieties selected from CN, phenyl, halogen, —C₁₋₄alkyl and —C₁₋₄alkoxy,
said process comprising:
reducing 3-benzyloxy-thiophene-2-carboxylic acid with hydrogen with palladium on carbon in ethanol solution;
reacting said 3-hydroxy-thiophene-2-carboxylic acid with an amine in dimethyl formamide in the presence of 2-(7-aza-1H-benzotriazole-1-yl)-1,1,3,3-tetramethyluronium hexafluorophosphate/triethanolamine to form an N-substituted 3-hydroxy-thiophene-2-carboxamide;
reacting said carboxamide an alkyl bromide or a substituted alkyl bromide in dimethylformamide in the presence of cesium carbonate to form said compound of Formula I.

6. A process for preparing a compound of Formula I, 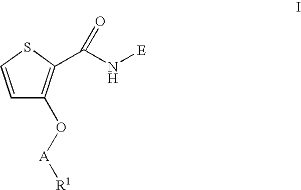
wherein:
E is selected from a moiety of formula II or III, 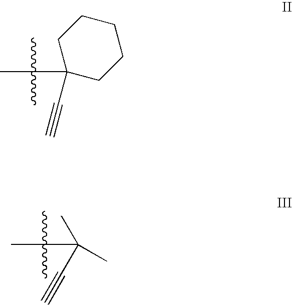
A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and
R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:
R¹ may be unsubstituted or be substituted with 1 or 2 moieties selected from CN, phenyl, halogen, —C₁₋₄alkyl and —C₁₋₄alkoxy,
said process comprising:
reacting an N-substituted 3-hydroxy-thiophene-2-carboxamide with an alkyl bromide or a substituted alkyl bromide in dimethylformamide in the presence of cesium carbonate to form said compound of Formula I.

7. A method of treatment or prophylaxis of a disease or condition in which modulation of the alpha 7 receptor is beneficial which method comprises administering to a subject suffering from said disease or condition a therapeutically-effective amount of a compound in accord with Formula I: 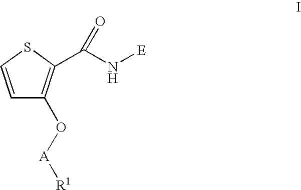
wherein:
E is selected from a moiety of formula II or III, 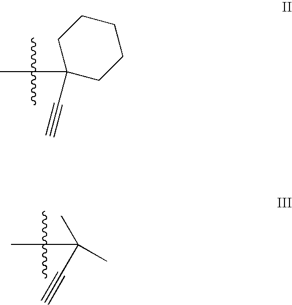
A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and
R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:
R¹ may be unsubstituted or be substituted with 1 or 2 moieties selected from CN, phenyl, halogen, —C₁₋₄alkyl and —C₁₋₄alkoxy,
or a stereoisomer, enantiomer, in vivo-hydrolysable precursor or pharmaceutically-acceptable salt thereof.

8. The method of claim 7, wherein said disease or condition is selected from Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, ulcerative colitis or irritable bowel syndrome.

9. A pharmaceutical composition comprising a pharmaceutically-acceptable diluent, lubricant or carrier and a compound in accord with Formula I: 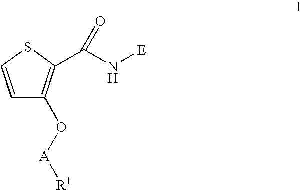
wherein:
E is selected from a moiety of formula II or III, 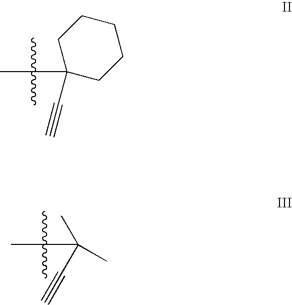
A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and
R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:
R¹ may be unsubstituted or be substituted with 1 or 2 moieties selected from CN, phenyl, halogen, —C₁₋₄alkyl and —C₁₋₄alkoxy,
or a stereoisomer, enantiomer, in vivo-hydrolysable precursor or pharmaceutically-acceptable salt thereof.

10. A method of treatment or prophylaxis of a disease or condition in which modulation of the alpha 7 receptor is beneficial which method comprises administering a therapeutically-effective amount of a pharmaceutical composition according to claim 9 to a subject suffering from said disease or condition.

11. The method of claim 10, wherein said disease or condition is selected from Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, ulcerative colitis or irritable bowel syndrome.

12. The use of a compound in accord with Formula I: 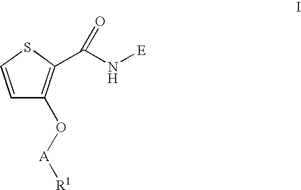
wherein:
E is selected from a moiety of formula II or III, 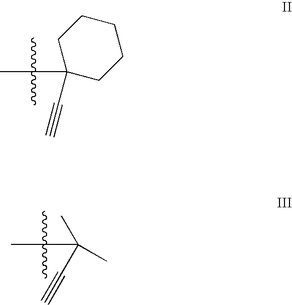
A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and
R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:
R¹ may be unsubstituted or be substituted with 1 or 2 moieties selected from CN, phenyl, halogen, —C₁₋₄alkyl and —C₁₋₄alkoxy,
or a stereoisomer, enantiomer, in vivo-hydrolysable precursor or pharmaceutically-acceptable salt thereof,
for the treatment or prophylaxis of a disease or condition in which modulation of the alpha 7 receptor is beneficial.

13. The use according to claim 12, wherein said disease or condition is selected from Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, ulcerative colitis or irritable bowel syndrome.

14. The use in the manufacture of a medicament for the treatment or prophylaxis of a disease or condition in which modulation of the alpha 7 receptor is beneficial of a compound in accord with Formula I: 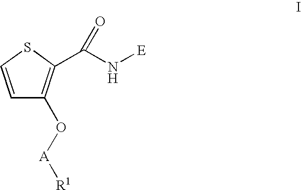
wherein:
E is selected from a moiety of formula II or III, 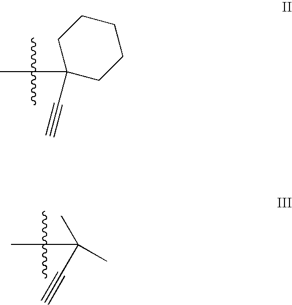
A is selected from —CH₂—C(═O)—, —(CH₂)₂—O—, —(CH₂)₂—O—CH₂—, —CH(—CH₃)— and —(CH₂)ₙ— where n is selected from 1, 2 or 3, and
R¹ is selected from H, CN, phenyl, pyridyl, isoxazolyl, triazolyl, dioxalanyl, benzoxadiazolyl, benzofuranyl, pyrazolyl, 1H-indolyl-C₁₋₆alkyl and —C₃₋₈cycloalkyl; wherein:
R¹ may be unsubstituted or be substituted with 1 or 2 moieties selected from CN, phenyl, halogen, —C₁₋₄alkyl and —C₁₋₄alkoxy,
or a stereoisomer, enantiomer, in vivo-hydrolysable precursor or pharmaceutically-acceptable salt thereof.

15. The use according to claim 14, wherein said disease or condition is selected from Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, ulcerative colitis or irritable bowel syndrome.
